(19) United States
(12) Patent Application Publication
(10) Pub. No.: US 2023/0330302 A1
(43) Pub. Date: Oct. 19, 2023

(54) ORTHOPEDIC REPAIR SCAFFOLD, PREPARATION METHOD THEREOF AND USE THEREOF

(71) Applicant: SHENZHEN INSTITUTES OF ADVANCED TECHNOLOGY CHINESE ACADEMY OF SCIENCES, Shenzhen, Guangdong (CN)

(72) Inventors: Yuxiao LAI, Shenzhen, Guangdong (CN); Yuanyi SUN, Shenzhen, Guangdong (CN)

(21) Appl. No.: 18/044,684

(22) PCT Filed: Jul. 30, 2021
(86) PCT No.: PCT/CN2021/109471

§ 371 Date: Mar. 9, 2023

(30) Foreign Application Priority Data
Jul. 23, 2021 (CN) 202110841435.9

(51) Int. Cl. A61L 27/44; A61L 27/58; A61L 27/56; A61L 27/02; A61F 2/30

(52) U.S. Cl. CPC A61L 27/446; A61L 27/58; A61L 27/56; A61L 27/025; A61F 2/3094; A61L 2430/02; A61L 2400/12; A61F 2002/30985; A61F 2310/00065

(57) ABSTRACT

Provided are an orthopedic repair scaffold, a preparation method thereof and use thereof. The orthopedic repair scaffold is a three-dimensional porous scaffold. A material of the orthopedic repair scaffold comprises the following components in mass percentage: 80%-95% of a biodegradable polymer and 5%-20% of a biodegradable nanoparticle, where the biodegradable nanoparticle is a nanoparticle of manganese compound. The preparation method of the orthopedic repair scaffold comprises: preparing a homogeneous solution comprising a biodegradable polymer and a biodegradable nanoparticle according to the mass percentage; preparing the homogeneous solution through a curing molding process into a molded three-dimensional porous scaffold; and freeze-drying the molded three-dimensional porous scaffold to obtain the orthopedic repair scaffold. The orthopedic repair scaffold can better promote healing of a bone injury and has an excellent mechanical performance and a good medical imaging function.

### CROSS-REFERENCE TO RELATED APPLICATIONS

The present application is a national phase entry under 35 USC § 371 of International Application PCT/CN2021/109471, filed Jul. 30, 2021, which claims the benefit of and priority to Chinese Patent Application No. 2021108414359, filed Jul. 23, 2021, the entire disclosures of which are incorporated herein by reference.

### TECHNICAL FIELD

The present disclosure relates to the field of biomedical engineering technologies, and particularly to an orthopedic repair scaffold, a preparation method thereof and use thereof.

### BACKGROUND

With the development of orthopedic treatment technology, a cure rate and therapeutic effect of clinical orthopedic diseases are persistently improved, but some bone defects cannot be healed on time due to various reasons. In addition, bone repair and regeneration processes are required for the treatment of bone defects associated with severe fractures, bone defects after surgery of tumors, and spinal fusion. A traditional autologous bone transplantation can basically meet the above requirements, has a reliable clinical curative effect, and is a gold standard for treating fracture nonunion and bone defects for a long time. However, autologous bone transplantation has problems about donor site complications, limited sources, etc., and therefore various alternative approaches to promote osseous repair and regeneration have been continuously explored and attempted. It is a research hotspot of a current tissue engineering scaffold to prepare an orthopedic repair scaffold with a biocompatibility, a degradability, and a good mechanical performance from a biodegradable polymer through printing.

As a typical biodegradable high polymer material, levorotatory polylactic acid (PLLA) has advantages of a wide source, a non-toxic degraded product, a good biocompatibility and the like. However, application of the orthopedic repair scaffold prepared from PLLA in the field of osseous repair is limited due to characteristics of a relatively insufficient mechanical strength, low medical imaging quality after the orthopedic repair scaffold is implanted into a defect site and the like.

### SUMMARY

In order to solve the problems that the orthopedic repair scaffold in the prior art is relatively insufficient in mechanical strength, low in medical imaging quality after implanted into a defect site and the like, the disclosure provides an orthopedic repair scaffold, a preparation method thereof and use thereof.

In order to achieve the above objects, the present disclosure adopts the following technical solution.

Provided is an orthopedic repair scaffold, where the orthopedic repair scaffold is a three-dimensional porous scaffold, a material of the orthopedic repair scaffold comprises the following components in mass percentage: 80%-95% of a biodegradable polymer and 5%-20% of a biodegradable nanoparticle, and the biodegradable nanoparticle is a nanoparticle of manganese compound.

Preferably, the manganese compound is selected from one or more than two of manganese dioxide, trimanganese tetraoxide, manganese gluconate, manganese chloride, manganese acetate, manganese dihydrogen phosphate, manganese carbonate, manganese sulfate, and manganese carbonyl.

Preferably, the nanoparticle of manganese compound has a particle size of 1 nm to 1,000 nm.

Preferably, the biodegradable polymer is selected from one or more than two of a polylactic acid-glycolic acid copolymer, polylactic acid, polylactic acid-glycolic acid, and polycaprolactone.

Preferably, a micropore in the three-dimensional porous scaffold has a pore diameter of 300 μm to 500 μm, and the three-dimensional porous scaffold has a porosity of 60%-80%.

Preferably, the micropore at least penetrates through two opposite surfaces of the three-dimensional porous scaffold.

In another aspect of the present disclosure, provide is a preparation method of the orthopedic repair scaffold, comprising the following steps:
preparing a homogeneous solution comprising a biodegradable polymer and a biodegradable nanoparticle according to the mass percentage;
preparing the homogeneous solution through a curing molding process into a molded three-dimensional porous scaffold; and
freeze-drying the molded three-dimensional porous scaffold to obtain the orthopedic repair scaffold.

Preferably, in the step of preparing a homogeneous solution comprising a biodegradable polymer and a biodegradable nanoparticle, the biodegradable polymer and the biodegradable nanoparticle are combined in a manner of stirring mixing or chemical reaction; and the curing molding process is a 3D printing molding process, a fused deposition molding process, a template molding process or a pore-forming agent adding molding process.

Preferably, the curing molding process is a 3D printing molding process, and the preparing the homogeneous solution through a curing molding process into a molded three-dimensional porous scaffold comprises the following steps:
creating a model using design software and acquiring corresponding printing parameters; and
adding the homogeneous solution into a 3D printing device, and performing printing and molding according to the printing parameters to obtain the molded three-dimensional porous scaffold,
wherein the printing parameters comprise: a spinning spacing of 0.4 mm to 2 mm, a printing layer height of 0.08 mm to 0.16 mm, a spray head moving rate of 1 mm/s to 20 mm/s, a spray head discharge rate of 0.1 mm³/s to 1 mm³/s, a printing temperature of −40° C. to −20° C., a freeze-drying temperature of −40° C. to −100° C., and a time of 24 h to 72 h.

The present disclosure further provides use of the orthopedic repair scaffold in osteogenesis and medical imaging.

The embodiments of the present disclosure provide an orthopedic repair scaffold and a preparation method thereof, which have the following beneficial effects:

(1) A material of the orthopedic repair scaffold is a biodegradable polymer containing a nanoparticle of manganese compound. The manganese compound can consume excessive hydrogen peroxide in a microenvironment and generate oxygen and a manganese ion which are beneficial for repair. When the material is applied to the treatment of a bone injury, the hydrogen peroxide at an injury site can be consumed to inhibit an inflammatory reaction, at the same time the oxygen content at the injury site can also be improved, so as to improve an activity of osteoblasts, and promote healing of a bone injury. Besides, a bone injury healing process can be further accelerated by an osteogenesis-promoting effect of the manganese ion.

(2) The orthopedic repair scaffold prepared by adding the nanoparticle of manganese compound has a higher compressive strength and compressive modulus and an excellent mechanical performance. Furthermore, the added manganese compound has an excellent CT imaging function and effectively improves a medical imaging effect of the orthopedic repair scaffold.

(3) The orthopedic repair scaffold is of a three-dimensional porous scaffold structure, has a high porosity, and is beneficial for growth, attachment, proliferation and the like of osteoblasts. Meanwhile, the porous structure of the scaffold can induce bone ingrowth. Therefore, the orthopedic repair scaffold has a great application value in the field of bone defect treatment for guiding bone regeneration.

### BRIEF DESCRIPTION OF THE DRAWINGS

FIG. 1 is a structural schematic diagram of the orthopedic repair scaffold prepared according to Embodiment 1 of the present disclosure;

FIG. 2 is a cross-sectional schematic diagram of the orthopedic repair scaffold prepared according to Embodiment 1 of the present disclosure;

FIG. 3 is a structural schematic diagram of the orthopedic repair scaffold prepared according to a Comparative Example;

FIG. 4 is an image illustration of CT imaging of a test sample A according to a Test Example; and

FIG. 5 is an image illustration of CT imaging of a test sample B according to a Test Example.

### DETAILED DESCRIPTION

To make the objectives, features and advantages of the disclosure more obvious and understandable, the specific embodiments of the disclosure will be described in detail in combination with drawings, but these embodiments are merely exemplary and cannot be construed as limitation to the implementable range of the disclosure.

It should be noted that, in order to avoid obscuring the disclosure with unnecessary details, only the structures and/or processing steps closely related to the solution according to the disclosure are shown in the drawings, and other details having little relation with the disclosure are omitted.

As described above, in order to solve the problems that an existing orthopedic repair scaffold prepared from a biodegradable polymer (e.g., PLLA) is a relatively insufficient in mechanical strength and low in medical imaging quality after implanted into a defect site, the embodiments of the present disclosure provides an orthopedic repair scaffold and a preparation method thereof. A nanoparticle of manganese compound is added into the biodegradable polymer, such that the orthopedic repair scaffold can better promote healing of a bone injury, and has an excellent mechanical performance and a good medical imaging effect.

The embodiments of the present disclosure firstly provide an orthopedic repair scaffold, where the orthopedic repair scaffold is a three-dimensional porous scaffold molded by printing and a material of the orthopedic repair scaffold comprises the following components in mass percentage:
80%-95% of a biodegradable polymer, such as 80%, 82%, 85%, 88%, 90%, 93% or 95% of the biodegradable polymer, and
5%-20% of a biodegradable nanoparticle, such as 5%, 7%, 10%, 12%, 15%, 18% or 20% of the biodegradable nanoparticle, wherein the biodegradable nanoparticle is a nanoparticle of manganese compound.

The material of the orthopedic repair scaffold is the biodegradable polymer containing the nanoparticle of manganese compound. The nanoparticle of manganese compound can consume excessive hydrogen peroxide in a microenvironment and generate oxygen and a manganese ion beneficial for repair, and when the material is applied to a treatment for the bone injury, the hydrogen peroxide at an injury site can be consumed to inhibit an inflammatory reaction, at the same time the oxygen content at the injury site can also be improved, so as to improve an activity of osteoblasts, and promote healing of a bone injury. Besides, a bone injury healing process can be further accelerated by an osteogenesis-promoting effect of the manganese ion.

Furthermore, the orthopedic repair scaffold prepared by adding the nanoparticle of manganese compound has a higher compressive strength and compressive modulus and an excellent mechanical performance. In addition, the added manganese oxide nanoparticle has an excellent CT imaging function and effectively improves a medical imaging effect of the orthopedic repair scaffold.

In a specific embodiment, the manganese compound is selected from one or more than two of manganese dioxide, trimanganese tetraoxide, manganese gluconate, manganese chloride, manganese acetate, manganese dihydrogen phosphate, manganese carbonate, manganese sulfate, and manganese carbonyl. Preferably, the nanoparticle is a nanoparticle of manganese oxide, for example, a particle of manganese dioxide or a particle of trimanganese tetraoxide. Preferably, the nanoparticle of manganese compound has a particle size of 1 nm to 1,000 nm, such as 1 nm, 5 nm, 10 nm, 50 nm, 100 nm, 200 nm, 300 nm, 500 nm, 800 nm or 1,000 nm, and more preferably, the nanoparticle of manganese compound has a particle size of 100 nm to 300 nm.

In a specific embodiment, the biodegradable polymer is selected from one or more than two of a polylactic acid-glycolic acid copolymer (PLGA), polylactic acid (PLA), polylactic acid-glycolic acid (PLGA), and polycaprolactone (PCL).

In a preferred embodiment, the biodegradable polymer is selected from levorotatory polylactic acid (PLLA) and the biodegradable nanoparticle is a particle of manganese dioxide. Further preferably, a material of the orthopedic repair scaffold comprises the following components in mass percentage: 95% of levorotatory polylactic acid and 5% of a particle of manganese dioxide.

In a specific embodiment, a micropore in the three-dimensional porous scaffold has a pore diameter of 300 μm to 500 μm, and the three-dimensional porous scaffold has a porosity of 60% to 80%. The orthopedic repair scaffold with the structure of a high porosity is beneficial for growth, attachment, proliferation and the like of osteoblasts. Meanwhile, the porous structure of the orthopedic repair scaffold can induce bone ingrowth.

In a preferable embodiment, the micropore at least penetrates through two opposite surfaces of the three-dimensional porous scaffold. For example, the micropore penetrates through the upper and lower surfaces of the three-dimensional porous scaffold in a height direction.

The embodiments of the present disclosure provide a preparation method of the orthopedic repair scaffold, comprising the following steps:

S10. Preparing a homogeneous solution comprising a biodegradable polymer and a biodegradable nanoparticle according to the above mass percentage.

In a specific embodiment, the biodegradable polymer and the biodegradable nanoparticle are combined in a manner of stirring mixing or chemical reaction.

The manner of stirring mixing is preferred. Specifically, the biodegradable polymer and the biodegradable nanoparticle are weighed according to the above mass percentage and dissolved into an organic solvent, and a homogeneous solution is obtained through stirring and mixing.

S20. Preparing the homogeneous solution through a curing molding process into a molded three-dimensional porous scaffold.

Specifically, the curing molding process is a 3D printing molding process, a fused deposition molding process, a template molding process or a pore-forming agent adding molding process, preferably the 3D printing process.

In a preferred embodiment, the curing molding process is a 3D printing process. In step S10 of preparing the homogeneous solution, the organic solvent may be selected from any one of the existing organic solvents favorable for 3D printing and molding. A dissolving process may be performed at a temperature of 40° C. to 60° C., preferably 55° C.

The preparing the homogeneous solution through a 3D printing process into a molded three-dimensional porous scaffold comprises the following steps:

S21. Creating a model using design software and acquiring corresponding printing parameters.

In a specific embodiment, a model is created by using BioMakerV2 software adapted to a low-temperature rapid molding device to obtain a three-dimensionally structural model; and data of the three-dimensionally structural model are exported, layering processing is performed on the data by using layering software to obtain layered data, and corresponding printing parameters are set according to the layered data.

In some preferred embodiments, the printing parameters comprise: a spinning spacing of 0.4 mm to 2 mm, a printing layer height of 0.08 mm to 0.16 mm, a spray head moving rate of 1 mm/s to 20 mm/s, a spray head discharge rate of 0.1 mm³/s to 1 mm³/s, and a printing temperature of −40° C. to −20° C. In a specific technical solution, the printing parameters comprise: a spinning spacing of 1 mm, a printing layer height of 0.12 mm, a spray head moving rate of 10 mm/s, a spray head discharge rate of 0.5 mm³/s, and a printing temperature of −30° C.

S22. Adding the homogeneous solution into a 3D printing device, and performing printing and molding according to the printing parameters to obtain the molded three-dimensional porous scaffold.

The three-dimensional porous scaffold molded by printing can be designed to the orthopedic repair scaffold with different structures according to shapes of bone defects. A micropore in the three-dimensional porous scaffold may have a pore diameter of 300 μm to 500 μm by adjusting the printing parameters, such as 300 μm, 350 μm, 400 μm, 450 μm or 500 μm. The three-dimensional porous scaffold has a porosity of 60%-80%.

S30. Freeze-drying the molded three-dimensional porous scaffold to obtain the orthopedic repair scaffold.

In some preferred embodiments, the freeze-drying temperature is −40° C. to −100° C. and a time is 24 h to 72 h.

In view of the fact that the orthopedic repair scaffold provided by the embodiments of the present disclosure has performances of promoting healing of a bone injury by improving an activity of osteoblasts and a good imaging function, the embodiments of the present disclosure further provide use of the orthopedic repair scaffold in osteogenesis and medical imaging (including CT, MRI, PET, etc.).

### Embodiment 1

A material of the orthopedic repair scaffold of the embodiment comprises the following components in mass percentage: 95% of PLLA and 5% of a nanoparticle of manganese dioxide.

A preparation method for the composite orthopedic repair scaffold comprised the following steps:

(1). 95% of PLLA and 5% of a nanoparticle of manganese dioxide with a particle size of 100 nm were weighed according to the mass percentage and placed in a beaker, then 1,4-dioxane was added, and the materials were stirred to form a homogeneous solution, where a concentration of the PLLA in the 1,4-dioxane was 0.125 g/mL.

(2). A model was created by using BioMakerV2 software adapted to a low-temperature rapid molding device to create a cubic structural model of 2×2×2 cm³; and data of the three-dimensionally structural model were exported and layering processing was performed on the data by using layering software to obtain layered data.

(3). The homogeneous solution obtained in step 1 was added into a material tank of the low-temperature rapid molding device, the device was assembled and the printing parameters were set according to the layered data in step 2, where a spinning spacing was 1 mm, a layer height was 0.12 mm, a spray head moving rate was 10 mm/s, and a spray head discharge rate was 0.5 mm³/s, and printing and molding were performed at −30° C. to obtain a three-dimensional porous scaffold of 2×2×2 cm³.

(4). The molded three-dimensional porous scaffold was freeze-dried in a freeze dryer at a temperature of −100° C. for 24 h to obtain an orthopedic repair scaffold.

FIG. 1 is a structural schematic diagram of the orthopedic repair scaffold prepared according to Embodiment 1. Since the nanoparticle of manganese dioxide with a mass percentage of 5% was added, the prepared orthopedic repair scaffold was light gray and had uniform pores and color.

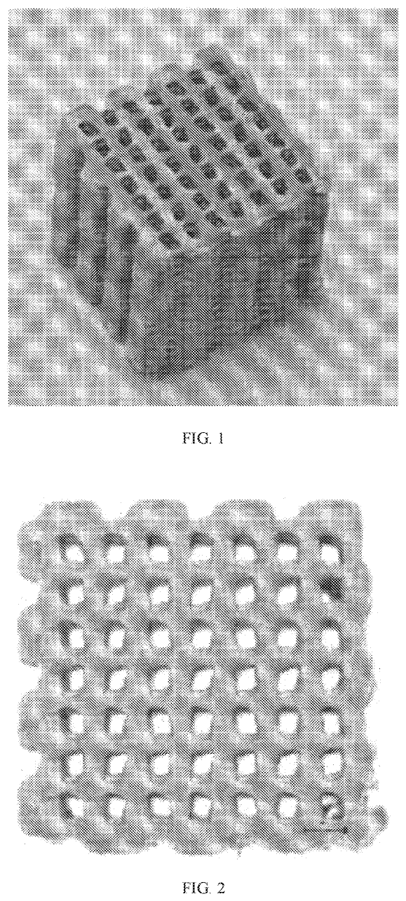

FIG. 2 is a cross-sectional schematic diagram of the orthopedic repair scaffold prepared according to Embodiment 1. A microscopic appearance observation of a cross section of the orthopedic repair scaffold was performed through a microscope. The orthopedic repair scaffold was provided with pores penetrating through upper and lower surfaces.

### Embodiment 2

A material of the orthopedic repair scaffold of the embodiment comprises the following components in mass percentage: 80% of PLA and 20% of a nanoparticle of manganese dioxide.

A preparation method for the composite orthopedic repair scaffold comprised the following steps:

(1). 80% of PLA and 20% of a nanoparticle of manganese dioxide with a particle size of 200 nm were weighed according to the mass percentages and placed in a beaker, then dimethyl sulfoxide (DMSO) was added, and the materials were stirred to form a homogeneous solution, where a concentration of the PLA in the DMSO was 0.1 g/mL.

(2). A model was created by using Solidworks software to create a cubic structural model of 3×3×3 cm³; and data of the three-dimensionally structural model were exported and layering processing was performed on the data by using layering software to obtain layered data.

(3). The homogeneous solution obtained in step 1 was added into a material tank of a low-temperature rapid molding device, the device was assembled and the printing parameters were set according to the layered data in step 2, where a spinning spacing was 1.1 mm, a layer height was 0.1 mm, a spray head moving rate was 20 mm/s, and a spray head discharge rate was 0.3 mm³/s, and printing and molding were performed at −25° C. to obtain a three-dimensional porous scaffold of 3×3×3 cm³.

(4). The molded three-dimensional porous scaffold was freeze-dried in a freeze dryer at a temperature of −85° C. for 48 h to obtain an orthopedic repair scaffold.

### Embodiment 3

A material of the orthopedic repair scaffold of the embodiment comprised the following components in mass percentage: 90% of PLGA and 10% of a nanoparticle of manganese dioxide.

A preparation method for the composite orthopedic repair scaffold comprises the following steps:

(1) 90% of PLGA and 10% of a nanoparticle of manganese dioxide with a particle size of 150 nm were weighed according to the mass percentages and placed in a beaker, then 1,4-dioxane was added, and the materials were stirred to form a homogeneous solution, where a concentration of the PLGA in the 1,4-dioxane was 0.15 g/mL.

(2) A model was created by using Solidworks software to create a cubic structural model of 2.5×2.5×2.5 cm³; and data of the three-dimensionally structural model were exported and layering processing was performed on the data by using layering software to obtain layered data.

(3) The homogeneous solution obtained in step 1 was added into a material tank of a low-temperature rapid molding device, the device was assembled and the printing parameters were set according to the layered data in step 2, where a spinning spacing was 1.2 mm, a layer height was 0.15 mm, a spray head moving rate was 15 mm/s, and a spray head discharge rate was 0.35 mm³/s, and printing and molding were performed at −28° C. to obtain a three-dimensional porous scaffold of 2.5×2.5×2.5 cm³.

(4) The molded three-dimensional porous scaffold was freeze-dried in a freeze dryer at a temperature of −80° C. for 48 h to obtain an orthopedic repair scaffold.

### Embodiment 4

A material of the orthopedic repair scaffold of the embodiment comprised the following components in mass percentage: 95% of PLLA and 5% of a manganese gluconate nanoparticle.

A preparation method for the composite orthopedic repair scaffold comprised the following steps:

(1) 95% of PLLA and 5% of a nanoparticle of manganese gluconate with a particle size of 100 nm were weighed according to the mass percentage and placed in a beaker, then 1,4-dioxane was added, and the materials were stirred to form a homogeneous solution, where a concentration of the PLLA in the 1,4-dioxane was 0.125 g/mL.

(2) A model was created by using BioMakerV2 software adapted to a low-temperature rapid molding device to create a cubic structural model of 2×2×2 cm³; and data of the three-dimensionally structural model were exported and layering processing was performed on the data by using layering software to obtain layered data.

(3) The homogeneous solution obtained in step 1 was added into a material tank of the low-temperature rapid molding device, the device was assembled and the printing parameters were set according to the layered data in step 2, where a spinning spacing was 1 mm, a layer height was 0.12 mm, a spray head moving rate was 10 mm/s, and a spray head discharge rate was 0.5 mm³/s, and printing and molding were performed at −30° C. to obtain a three-dimensional porous scaffold of 2×2×2 cm³.

(4) The molded three-dimensional porous scaffold was freeze-dried in a freeze dryer at a temperature of −80° C. for 24 h to obtain an orthopedic repair scaffold.

### Embodiment 5

A material of the orthopedic repair scaffold of the embodiment comprised the following components in mass percentages: 95% of PLLA and 5% of a manganese chloride nanoparticle.

A preparation method for the composite orthopedic repair scaffold comprises the following steps:

(1) 95% of PLLA and 5% of a manganese chloride nanoparticle with a particle size of 100 nm were weighed according to the mass percentage and placed in a beaker, then 1,4-dioxane was added, and the materials were stirred to form a homogeneous solution, where a concentration of the PLLA in the 1,4-dioxane was 0.125 g/mL.

(2) A model was created by using BioMakerV2 software adapted to a low-temperature rapid molding device to create a cubic structural model of 2×2×2 cm³; and data of the three-dimensionally structural model were exported and layering processing was performed on the data by using layering software to obtain layered data.

(3) The homogeneous solution obtained in step 1 was added into a material tank of the low-temperature rapid molding device, the device was assembled and the printing parameters were set according to the layered data in step 2, wherein a spinning spacing was 1 mm, a layer height was 0.12 mm, a spray head moving rate was 10 mm/s, and a spray head discharge rate was 0.5 mm³/s, and printing and molding were performed at −30° C. to obtain a three-dimensional porous scaffold of 2>2×2 cm³.

(4) The molded three-dimensional porous scaffold was freeze-dried in a freeze dryer at a temperature of −80° C. for 24 h to obtain an orthopedic repair scaffold.

### Comparative Example

The Comparative Example differs from Embodiment 1 in that: a material of an orthopedic repair scaffold only contains PLLA without adding a nanoparticle of manganese dioxide. The rest materials are the same and the rest process is performed according to Embodiment 1 to prepare the orthopedic repair scaffold sample of the Comparative Example.

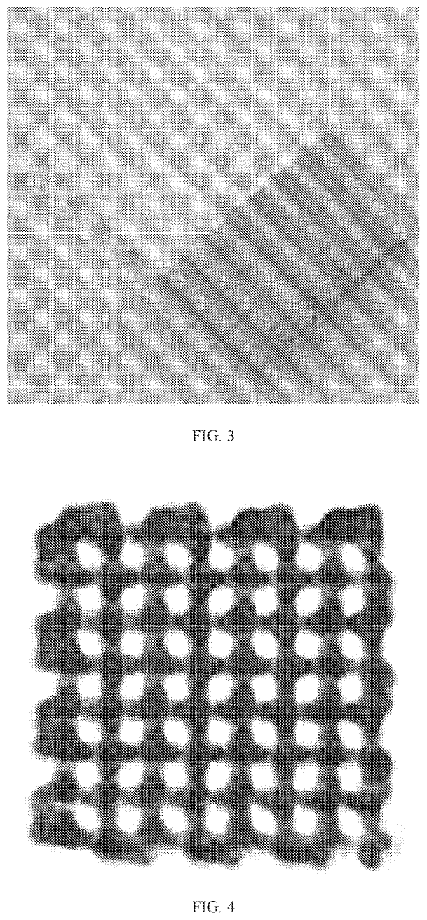

FIG. 3 is a structural schematic diagram of the orthopedic repair scaffold prepared according to the Comparative Example. Since no manganese oxide nanoparticle was added, the prepared orthopedic repair scaffold was in an original color of the PLLA (white) with uniform pores.

Test Example

Test samples A1-A5 and B were prepared according to Embodiments 1-5 and Comparative Example 1 respectively.

A difference between the test sample A1 and the orthopedic repair scaffold sample in Embodiment 1 was that a sample size was increased to 10×10×10 cm³. A difference between the test sample A2 and the orthopedic repair scaffold sample in Embodiment 2 was that a sample size was increased to 10×10×10 cm³. A difference between the test sample A3 and the orthopedic repair scaffold sample in Embodiment 3 was that a sample size was increased to 10×10×10 cm³. A difference between the test sample A4 and the orthopedic repair scaffold sample in Embodiment 4 was that a sample size was increased to 10×10×10 cm³. A difference between the test sample A5 and the orthopedic repair scaffold sample in Embodiment 5 was that a sample size was increased to 10×10×10 cm³. A difference between the test sample B and the orthopedic repair scaffold sample in the Comparative Example was that a sample size was increased to 10×10×10 cm³.

The test samples were tested as follows:

(1) Test of Compressive Strength and Compressive Modulus

A test was performed by using a universal mechanical tester with a compression rate of 1 mm/min. 4 samples were selected each from the test samples A1-A5 and B respectively. Test results were averaged. The specific test results were shown in Table 1.

TABLE 1

| | Parameters | |
|---|---|---|
| | Compressive | Compressive |
| | strength | modulus |
| Samples | (MPa) | (MPa) |
| Test sample A1 | 2.5475 ± 0.2902 | 37.8338 ± 2.2803 |
| Test sample A2 | 2.2658 ± 0.3527 | 40.0883 ± 1.24361 |
| Test sample A3 | 3.092 ± 0.5329 | 39.2841 ± 0.29401 |
| Test sample A4 | 2.9906 ± 0.6772 | 41.93371 ± 2.97137 |
| Test sample A5 | 2.447 ± 0.3661 | 35.12852 ± 2.3351 |
| Test sample B | 1.765 ± 0.08201 | 30.61358 ± 0.89211 |

It can be seen from data in Table 1 that prior to adding a nanoparticle of manganese compound, the compressive strength of the orthopedic repair scaffold was about 1.7 MPa and the compressive modulus was about 30 MPa; and after the nanoparticle of manganese compound was added, the compressive strength and the compressive modulus of the orthopedic repair scaffold were both greatly improved. It can be seen that the nanoparticle of manganese compound can obviously improve the compressive strength and the compressive modulus of the orthopedic repair scaffold. Therefore, the orthopedic repair scaffold provided according to the embodiments of the present disclosure has a better mechanical performance and can play a better supporting role in bone filling.

(2) Test of Medical Imaging Effect

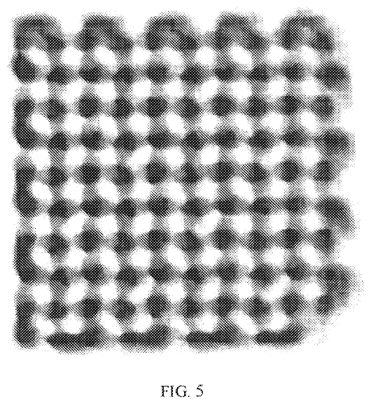

The test samples A1 and B were respectively scanned by micro computed tomography (micro-CT), and reconstructed and analyzed by using CT-Analyser software built in a system. FIG. 4 is an exemplary image of CT imaging of the test sample A1, and FIG. 5 is an exemplary image of CT imaging of the test sample B. It can been seen by comparing FIGS. 4 and 5 that under the same scanning and reconstruction parameters, the test sample A1 had a clearer imaging effect than the test sample B. Therefore, the orthopedic repair scaffold provided according to the embodiments of the present disclosure has a better medical imaging effect and is clearer in imaging under CT.

In conclusion, according to the orthopedic repair scaffold and the preparation method thereof provided according to the embodiments of the present disclosure, the nanoparticle of manganese compound was added to the biodegradable polymer, such that the orthopedic repair scaffold can better promote healing of a bone injury and has an excellent mechanical performance and a good medical imaging effect.

The above embodiments with specific and detailed description merely represent several embodiments according to the present disclosure, and it should be understood that for those of ordinary skill in the art, variations and modifications can be made without departing from the concept of the present disclosure, all of which fall within the scope of the present disclosure.

1. An orthopedic repair scaffold, wherein the orthopedic repair scaffold is a three-dimensional porous scaffold, a material of the orthopedic repair scaffold comprises the following components in mass percentage: 80%-95% of a biodegradable polymer and 5%-20% of a biodegradable nanoparticle, and the biodegradable nanoparticle is a nanoparticle of manganese compound.

2. The orthopedic repair scaffold according to claim 1, wherein the manganese compound is selected from one or more than two of manganese dioxide, trimanganese tetraoxide, manganese gluconate, manganese chloride, manganese acetate, manganese dihydrogen phosphate, manganese carbonate, manganese sulfate, and manganese carbonyl.

3. The orthopedic repair scaffold according to claim 1, wherein the nanoparticle of manganese compound has a particle size of 1 nm to 1,000 nm.

4. The orthopedic repair scaffold according to claim 1, wherein the biodegradable polymer is selected from one or more than two of a polylactic acid-glycolic acid copolymer, polylactic acid, polylactic acid-glycolic acid, and polycaprolactone.

5. The orthopedic repair scaffold according to claim 1, wherein a micropore in the three-dimensional porous scaffold has a pore diameter of 300 μm to 500 μm, and the three-dimensional porous scaffold has a porosity of 60%-80%.

6. The orthopedic repair scaffold according to claim 5, the micropore at least penetrates through two opposite surfaces of the three-dimensional porous scaffold.

7. A method for the preparation of an orthopedic repair scaffold, comprising:
preparing a homogeneous solution comprising 80%-95% of a biodegradable polymer and 5%-20% of a biodegradable nanoparticle according to a mass percentage, wherein the biodegradable nanoparticle is a nanoparticle of manganese compound;
preparing the homogeneous solution through a curing molding process into a molded three-dimensional porous scaffold; and
freeze-drying the molded three-dimensional porous scaffold to obtain the orthopedic repair scaffold.

8. The method according to claim 7, wherein in the step of preparing the homogeneous solution, the biodegradable polymer and the biodegradable nanoparticle are combined in a manner of stirring mixing or chemical reaction; and the curing molding process is a 3D printing molding process, a fused deposition molding process, a template molding process or a pore-forming agent adding molding process.

9. The method according to claim 8, wherein the curing molding process is a 3D printing molding process, and the preparing the homogeneous solution through a curing molding process into a molded three-dimensional porous scaffold comprises the following steps:
creating a model using design software and acquiring corresponding printing parameters; and
adding the homogeneous solution into a 3D printing device, and performing printing and molding according to the printing parameters to obtain the molded three-dimensional porous scaffold,
wherein in the printing parameters comprise: a spinning spacing of 0.4 mm to 2 mm, a printing layer height of 0.08 mm to 0.16 mm, a spray head moving rate of 1 mm/s to 20 mm/s, a spray head discharge rate of 0.1 mm³/s to 1 mm³/s, a printing temperature of −40° C. to −20° C., a freeze-drying temperature of −40° C. to −100° C., and a time of 24 h to 72 h.

10. The method according to claim 7, wherein the manganese compound is selected from one or more than two of manganese dioxide, trimanganese tetraoxide, manganese gluconate, manganese chloride, manganese acetate, manganese dihydrogen phosphate, manganese carbonate, manganese sulfate, and manganese carbonyl.

11. The method according to claim 7, wherein the nanoparticle of manganese compound has a particle size of 1 nm to 1,000 nm.

12. The method according to claim 11, wherein the nanoparticle of manganese compound has a particle size of 100 nm to 300 nm.

13. The method according to claim 7, wherein the biodegradable polymer is selected from one or more of a polylactic acid-glycolic acid copolymer, polylactic acid, polylactic acid-glycolic acid, and polycaprolactone.

14. The method according to claim 7, wherein a micropore in the three-dimensional porous scaffold has a pore diameter of 300 μm to 500 μm, and the three-dimensional porous scaffold has a porosity of 60%-80%.

15. A method for osteogenesis and medical imaging, comprising using an orthopedic repair scaffold, wherein the orthopedic repair scaffold is a three-dimensional porous scaffold, a material of the orthopedic repair scaffold comprises the following components in mass percentage: 80%-95% of a biodegradable polymer and 5%-20% of a biodegradable nanoparticle, and the biodegradable nanoparticle is a nanoparticle of manganese compound.

16. The method according to claim 15, wherein the manganese compound is selected from one or more than two of manganese dioxide, trimanganese tetraoxide, manganese gluconate, manganese chloride, manganese acetate, manganese dihydrogen phosphate, manganese carbonate, manganese sulfate, and manganese carbonyl.

17. The method according to claim 15, wherein the nanoparticle of manganese compound has a particle size of 1 nm to 1,000 nm.

18. The method according to claim 15, wherein the biodegradable polymer is selected from one or more than two of a polylactic acid-glycolic acid copolymer, polylactic acid, polylactic acid-glycolic acid, and polycaprolactone.

19. The method according to claim 15, wherein a micropore in the three-dimensional porous scaffold has a pore diameter of 300 μm to 500 μm, and the three-dimensional porous scaffold has a porosity of 60%-80%.

20. The method according to claim 15, wherein the micropore at least penetrates through two opposite surfaces of the three-dimensional porous scaffold.
